(19) United States
(12) Patent Application Publication
(10) Pub. No.: US 2020/0155714 A1
(43) Pub. Date: May 21, 2020

(54) DUAL-TARGET IMAGING MOLECULAR PROBE, PREPARATION METHOD THEREFOR, AND APPLICATIONS THEREOF

(71) Applicant: PEKING UNION MEDICAL COLLEGE HOSPITAL, CHINESE ACADEMY OF MEDICAL SCIENCES, Beijing (CN)

(72) Inventors: Zhaohui ZHU, Beijing (CN); Shaobo YAO, Beijing (CN)

(21) Appl. No.: 16/605,134

(22) PCT Filed: Apr. 12, 2018
(86) PCT No.: PCT/CN2018/082782

§ 371 Date: Oct. 14, 2019

(30) Foreign Application Priority Data
Apr. 17, 2017 (CN) 201710250652.4

(51) Int. Cl. A61K 51/08; A61K 51/06; A61B 6/03

(52) U.S. Cl. CPC A61K 51/082; A61K 51/083; A61B 6/037; A61K 51/065; A61K 51/088

(57) ABSTRACT

The present invention discloses a targeting polypeptide compound having dual targets, comprising a TATE cyclic peptide structure, an RGD cyclic peptide structure and a NOTA chelating group, wherein the TATE cyclic peptide structure, the RGD cyclic peptide structure and the NOTA chelating group are respectively linked by a PEG segment having a polymerization degree of 1 to 5 or directly linked to a same glutamic acid; the structure of the polypeptide compound can be represented as NOTA-PEGn-Glu{PEGm-TATE}-PEGP-RGD, where m, n and p are an integer from 0 to 5 respectively. The present invention further discloses a TATE-RGD dual-target radioactive molecular probe based on the polypeptide compound. The TATE-RGD dual-target polypeptide drug of the present invention may simultaneously bind to SSTR, integrin αvβ3, has higher receptor binding affinity and uptake, more excellent non-target tissue clearance rate, and better in vivo and in vitro stability.

### TECHNICAL FIELD

The present invention relates to a field of radioactive probes for disease diagnosis and treatment, in particular to a TATE-RGD dual-target radioactive molecular probe targeting integrin αᵥβ₃ and/or somatostatin receptor (SSTR) positive diseases and a preparation method thereof, and applications of the compound as targeting molecules for diagnosis and treatment.

### BACKGROUND ART

An important part of tumor growth is neovascularization, which is regulated by a variety of protein molecules. One of the key proteins is integrinαᵥβ₃, an extracellular matrix receptor, which is a heterodimeric transmembrane glycoprotein composed of two subunits. Integrin αᵥβ₃ is one of the important molecular markers of tumors, which is highly expressed on the surface of neovascular endothelial cells and some tumor cells, such as neuroblastoma, osteosarcoma, glioblastoma, breast cancer and prostate cancer; while Integrin αᵥβ₃ is not expressed or expressed very low in formed blood vessels and normal tissues. Due to its high expression in tumor growth and metastasis, integrin αᵥβ₃ has become one of the targets for diagnosis and treatment. Somatostatin (SST) functions through the somatostatin receptor (SSTR). SSTR belongs to a family of G protein-coupled receptors and is a glycoprotein with seven transmembrane domains. There are five different molecular subtypes for SSTR gene, namely SSTR1˜5. SSTR is distributed extensively. Many normal cells including endocrine cells and lymphocytes express SSTR, but its expression is more common in tumors such as gastrointestinal pancreatic neuroendocrine tumors (gastrinoma, insulinoma, glucagonoma), carcinoid, pituitary adenoma, pheochromocytoma, paraganglioma and medullary thyroid carcinoma than that in the normal tissues. SSTR plays an important role in the occurrence and development of a variety of human tumors.

SSTR is found in a variety of neuroendocrine tumors (NET), such as gastrointestinal pancreatic neuroendocrine tumors (gastrinoma, insulinoma, glucagonoma), carcinoid, pituitary adenoma, pheochromocytoma, paraganglioma, medullary thyroid carcinoma, etc. Radionuclide-labeled somatostatin analogues of ^{99m}Tc and ¹¹¹In, such as ^{99m}Tc-HYNIC-octreotide and ¹¹¹In-DTPA-octreotide, have been used in clinic for a long time and played important roles in the diagnosis and treatment of NET. In addition, the new somatostatin derivatives are also constantly emerging or improved, such as octreotide, TATE, etc., of which, TATE has a higher affinity for SSTR2 and most NETs express more SSTR2. Internationally, ⁶⁸Ga-DOTA-TATE related preclinical research has been fully carried out and ⁶⁸Ga-DOTA-TATE has been applied in clinical practice in Germany and some other countries in the world. Numerous studies have shown its superiority of the diagnosis and treatment guidance for gastrointestinal pancreatic endocrine tumor, pituitary adenoma, pheochromocytoma, paraganglioma, medullary thyroid carcinoma and small cell lung cancer, and other neuroendocrine tumors.

The arginine-glycine-aspartate (RGD) peptide-based nuclide tracer was developed in the 1980s. It can bind with the integrin αᵥβ₃ subunit that is highly expressed on the surface of neovascular endothelial cells and tumor cells, to display the tumor and suggest the degree of tumor neovascularization. SPECT (/CT) imaging or PET (/CT) imaging with ^{99m}Tc or ¹⁸F-labeled RGD peptides has been used in clinic in Western countries such as Europe and the United States. Some initial clinical applications are also carried out in a few units in china, suggesting that it has high safety and efficacy and is valuable for the diagnosis, staging and efficacy evaluation of a variety of tumors including breast cancer, liver cancer and glioma, etc.

The positron-emitting radionuclide-labeled peptides ⁶⁸Ga-DOTA-RGD and ⁶⁸Ga-DOTA-TATE have higher sensitivity and specificity to the receptor; since integrin αᵥβ₃ and SSTR2 receptors are highly expressed in most malignant tumors and benign NET and the PET imaging has high resolution, they can quantify the absorption of tumors and organs and have better superiority than SPECT. Meanwhile, the positron-based radionuclide ⁶⁸Ga³⁺⁶⁰ is produced by a generator, with the advantages of simple production process and low cost, so the PET/CT imaging with ⁶⁸Ga labeled drugs is necessary and will have higher clinical application value, to improve the detection rate of tumors; in addition, it has higher clinical significance in tumor staging and prognosis evaluation, tumor-directed surgery and therapeutic evaluation.

### SUMMARY OF THE INVENTION

The object of the present invention is to design and synthesize a TATE-RGD dual-target radioactive molecular probe and a preparation method thereof. The molecular probe contains two polypeptides of TATE and RGD for binding two targets on tumor cells, which is linked by PEG (PEG=polyethylene glycol) molecule, playing a role of adjusting the distance between functional groups and the pharmacokinetic properties in vivo. Compared with single-target molecular probes, this probe has higher tumor uptake and can achieve better in vivo imaging effect. The molecule can be radiolabeled by a bifunctional chelating agent NOTA-labeled radionuclide. In vivo, the radiolabeled probe is concentrated to the lesion by TATE and/or RGD polypeptide tumor cell receptor targeting, and perform imaging diagnosis and treatment of the lesions with highly expressed SSTR2 and/or integrin αᵥβ₃ using nucleus medical positron emission tomography.

The object of the invention is achieved by the following technical solutions:

First, the present invention provides a targeting polypeptide compound having dual targets, comprising a TATE cyclic peptide structure, an RGD cyclic peptide structure and a NOTA chelating group, wherein the TATE cyclic peptide structure, the RGD cyclic peptide structure and the NOTA chelating group are respectively linked by a PEG segment having a polymerization degree of 1 to 5 or directly linked to a same glutamic acid molecule; the structure of the polypeptide compound can be simplified as NOTA-PEGₙ-Glu{PEGₘ-TATE}-PEG_{P}-RGD, where m, n and p are an integer from 0 to 5 respectively.

In a preferred polypeptide compound herein, the TATE cyclic peptide structure, the NOTA chelating group and the RGD cyclic peptide structure are linked to a same glutamic acid molecule by a PEG segment having a degree of polymerization of 2 to 5 respectively.

In a further preferred polypeptide compound herein, the TATE cyclic peptide structure, the NOTA chelating group and the RGD cyclic peptide structure are linked to the same glutamic acid molecule by a PEG₄ segment respectively.

In a further preferred polypeptide compound herein, the TATE cyclic peptide structure and the RGD cyclic peptide structure are linked to two carboxyl terminals of the same glutamic acid molecule by a PEG₄ molecular segment respectively to form a stable amide bond; the NOTA chelating group is linked to the amino terminal of the same glutamic acid molecule by a PEG₄ segment; the polypeptide compound is designated as NOTA-3PEG₄-TATE-RGD, and the specific structure thereof is as shown in the formula (I) below:

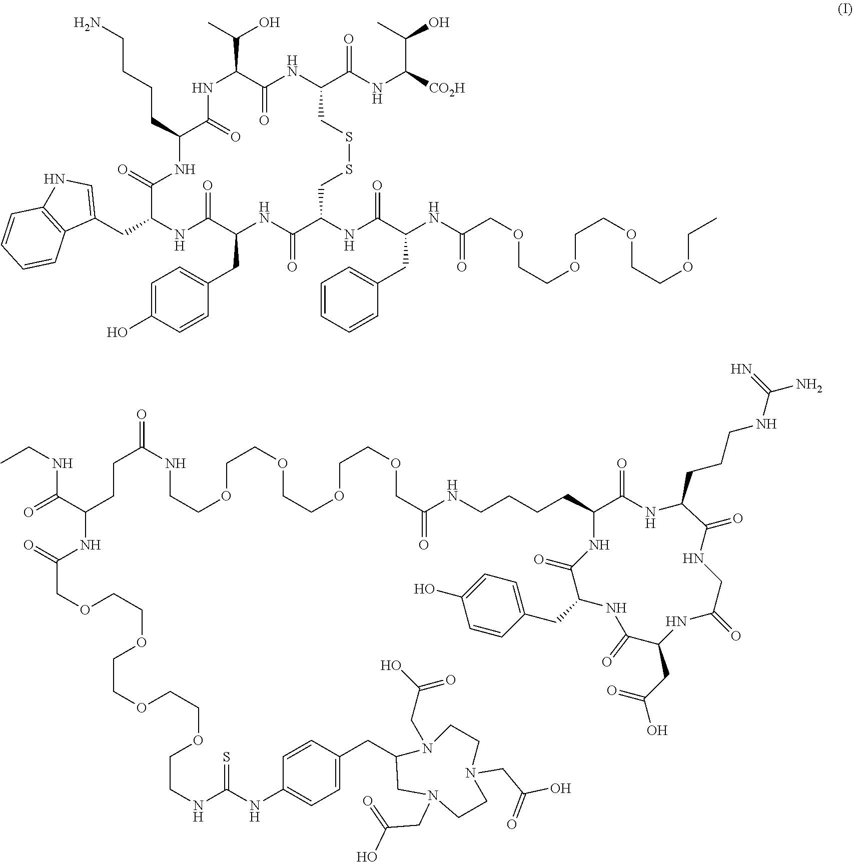

The present invention further provides a TATE-RGD dual-target radioactive molecular probe, a radionuclide-labeled polypeptide complex, wherein the polypeptide complex takes the targeting polypeptide compound having dual targets of the invention as a ligand.

In a preferred TATE-RGD dual-target radioactive molecular probe herein, the radionuclide is selected from any one of ⁶⁸Ga, ⁶⁴Cu, ¹⁸F, ⁸⁹Zr or ¹⁷⁷Lu; further preferably from any one of ⁶⁸Ga, ⁶⁴Cu or ¹⁸F. In a preferred embodiment of the invention, the TATE-RGD dual-target radioactive molecular probe is a ⁶⁸Ga-labeled polypeptide complex, the polypeptide complex takes the targeting polypeptide compound having dual targets of the invention as a ligand, and the TATE cyclic peptide structure, the RGD cyclic peptide structure and the NOTA chelating group of the dual-target tumor targeting polypeptide compound are linked to a same glutamic acid molecule by a PEG segment having a polymerization degree of 2 to 5.

In a most preferred embodiment of the invention, the TATE-RGD dual-target radioactive molecular probe is a ⁶⁸Ga-labeled polypeptide complex, the polypeptide complex takes the targeting polypeptide compound NOTA-3PEG₄-TATE-RGD having dual targets of the invention as a ligand, and the dual-target radioactive molecular probe is simply represented as ⁶⁸Ga-NOTA-3PEG₄-TATE-RGD.

Another object of the invention is to provide a method for preparing the polypeptide compound and the dual-target radioactive molecular probe, which is easy and feasible, with a high stability of product.

The above object of the present invention is achieved by the following technical solutions:

1. A method of preparing a tumor targeting polypeptide compound having dual targets, comprising the following steps:
a) mixing a protected glutamic acid with polypeptide PEGₙ-TATE in a molar ratio of 1-10:1-10, and carrying out an amino condensation reaction to obtain a first product linked by PEGₙ segment of TATE polypeptide and the protected glutamic acid, where n is an integer from 0 to 5;
b) deprotecting the group Fmoc of the first product obtained in the step a) under the piperidine condition to obtain a second product, simply represented as Glu-PEGₙ-TATE, wherein n is an integer from 0 to 5;
c) reacting the second product obtained in step b) with NOTA-PEGₘ-NHS under DIPEA condition to obtain a third product of Boc-protected glutamic acid that is linked to the NOTA group and the TATE peptide by a PEGₘ segment and a PEGₙ segment respectively, wherein n and m are integers from 0 to 5 respectively;
d) deprotecting the group Fmoc of the third product obtained in the step c) under the TFA conditions to obtain a fourth product of glutamic acid that is linked to the NOTA group and the TATE peptide by a PEGₘ segment and a PEGₙ segment respectively, simply represented as NOTA-PEGₘ-Glu(PEGₙ-TATE), where n and m are integers from 0 to 5 respectively;
e) reacting the fourth product obtained in step d) with the polypeptide PEG_{P}-RGD under the DIPEA condition, where p is an integer of 0 to 5, finally obtaining a tumor targeting polypeptide compound having dual targets NOTA-PEGₙ-Glu{PEGₘ-TATE}-PEG_{P}-RGD.

2. A method of preparing TATE-RGD dual-target radioactive molecular probe, taking the preparation of ⁶⁸Ga-NOTA-3PEG₄-TATE-RGD radiopharmaceutical as an example, comprising the following steps:
dissolving the NOTA-3PEG₄-TATE-RGD in deionized water; rinsing germanium-gallium (⁶⁸Ge/, ⁶⁸Ga) generator into a EP tube with 5 mL of 0.1 mol/L high-purity hydrochloric acid solution, collecting 1 mL of the solution containing the highest content of radioactivity, adding 93 μL of 1.25 mol/L sodium acetate to adjust the pH of the mixture to 4-4.5; adding 20 μg of the precursor to the mixture and mix well, heat to 100° C. for 10 min; after completion of the reaction, cooling the reaction solution to room temperature, then adding 4 mL of sterile water for injection, filtering the solution to a sterile product bottle through a sterile filter membrane (0.22 μm, 13 mm).

The ⁶⁸Ga-NOTA-3PEG₄-TATE-RGD radioactivity detection method, including:
analytical HPLC method, using high performance liquid chromatographic instrument (Waters, USA, Model 515 pump); UV detector (Model 486, UV absorption wavelength=254 nm), radioactivity detector (US EG&G BERTHOLD), radioactivity meter CRC-25 PET (Capintec, USA), Waters column Nova-Pak C18 (4.6×150 mm, 5 μm);

Further, the analytical HPLC method in the previous step is carried out using a Waters HPLC system equipped with a Waters C18 analytical column (4.6 mm×250 mm), HPLC gradient elution conditions: 0 min, acetonitrile/water (5/95, v/v); 5 min, acetonitrile/water (5/95, v/v); 10 min, acetonitrile/water (80/20, v/v); 15 min, acetonitrile/water (100/0, v/v); 18 min , acetonitrile/water (100/0, v/v); 20 min, acetonitrile/water (5/95, v/v), 0.1% TFA in the eluent, flow rate of 1 mL/min. From the radioactive HPLC profile, the retention time of ⁶⁸Ga-NOTA-3PEG4-TATE-RGD is 11.6 min, and the radiochemical purity is >99%.

The invention further provides applications of TATE-RGD dual-target molecular probe in the preparation of radiopharmaceuticals for SSTR2 and/or integrin αvβ3 positive lesion imaging diagnosis.

The preferred application of the invention is that the TATE-RGD dual-target molecular probe is prepared as a colorless transparent injection for small cell lung cancer imaging diagnosis.

Compared with the prior art, the TATE-RGD dual-target molecular probe has the following beneficial effects.
1. The TATE-RGD of the present invention is a dual-target polypeptide drug, and TATE and RGD are linked to enable simultaneous binding to SSTR and integrin αᵥβ_{3,} which increases binding affinity and tumor uptake of drugs, and achieves better tumor imaging effect;
2. In the present invention, by introducing a PEG molecule having the same or different degree of polymerization between the TATE and the RGD polypeptide, it can improve the pharmacokinetic properties of the polypeptide drug, especially the non-tumor tissue clearance rate;
3. The use of NOTA as a chelating agent in the present invention has better in vivo and in vitro stability than that of DOTA.

### BRIEF DESCRIPTION OF THE DRAWINGS

FIG. 1 shows ⁶⁸Ga-NOTA-3PEG₄-TATE-RGD HPLC pattern.

FIG. 2 shows a microPET imaging result of small cell lung cancer H69 model of nude mice. From the left to the right, the imaging results of injection with ⁶⁸Ga-NOTA-3PEG₄-TATE-RGD (Control), ⁶⁸Ga-NOTA-3PEG₄-TATE-RGD+RGD (Block+RGD), ⁶⁸Ga-NOTA-3PEG₄-TATE-RGD+TATE (Block+TATE) and ⁶⁸Ga-NOTA-3PEG₄-TATE-RGD+RGD+TATE (Block+TATE+RGD) from tail veins, respectively.

FIG. 3 shows a microPET imaging result of non-small cell lung cancer A549 model of nude mice. From the left to the right, the imaging results of injection with ⁶⁸Ga-NOTA-3PEG₄-TATE-RGD (Control), ⁶⁸Ga-NOTA-3PEG₄-TATE-RGD+RGD (Block+RGD),⁶⁸Ga-NOTA-3PEG₄-TATE-RGD+TATE(Block+TATE) and ⁶⁸Ga-NOTA-3PEG₄-TATE-RGD+RGD+TATE (Block+TATE+RGD) from tail veins, respectively.

FIG. 4 shows the radioactive uptake % ID/g of each organ at different time points after injection of ⁶⁸Ga-NOTA-3PEG₄-TATE-RGD in normal mice.

FIG. 5 shows comparison of imaging results of ⁶⁸Ga-NOTA-3PEG₄-TATE-RGD of the present invention with the existing ⁶⁸Ga-NOTA-RGD probe in patients with non-small cell lung cancer, wherein the left figure is the imaging result of ⁶⁸Ga-NOTA-3PEG₄-TATE-RGD of the invention, and the right figure is the imaging result of existing ⁶⁸Ga-NOTA-RGD, and the arrow points to the lesion.

FIG. 6 shows comparison of imaging results of ⁶⁸Ga-NOTA-3PEG₄-TATE-RGD with the existing ⁶⁸Ga-NOTA-RGD probe in patients with small cell lung cancer, wherein the left figure is the imaging result of ⁶⁸Ga-NOTA-3PEG₄-TATE-RGD of the invention, and the right figure is the imaging result of existing ⁶⁸Ga-NOTA-RGD, and the arrow points to the lesion.

### DETAILED DESCRIPTION OF THE PREFERRED EMBODIMENTS

In order to explain the technical solutions of the present invention more clearly, the present invention will be further illustrated and described below with reference to the accompanying drawings and embodiments, but the technical solutions of the present invention are not limited to the specific embodiments and some examples described below. The chemical products and reagents used in the following embodiments herein are existing or commercially available products.

Embodiment 1

A targeting polypeptide compound having dual targets, comprising a TATE cyclic peptide structure, an RGD cyclic peptide structure and a NOTA chelating group, formed by linking the same glutamic acid molecule by the TATE cyclic peptide structure, the NOTA chelating group and the RGD cyclic peptide structure respectively, wherein the TATE cyclic peptide structure and the RGD cyclic peptide structure are linked to the two carboxyl terminals of the glutamic acid molecule through a PEG4 molecular segment respectively to form a stable amide bond; the NOTA chelating group is linked to the amino terminal of the glutamic acid molecule by a PEG4 molecule; the obtained polypeptide compound is designated as NOTA-3PEG₄-TATE-RGD, and the specific structure thereof is as shown in the formula (I) below:

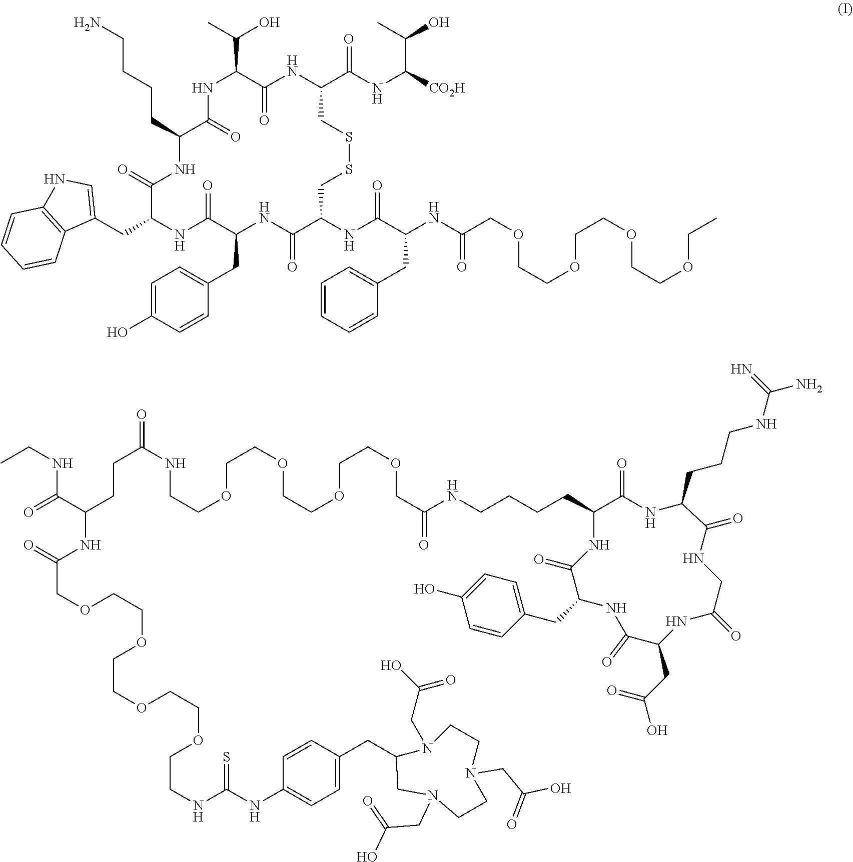

A method of preparing the NOTA-3PEG₄-TATE-RGD comprises the following steps:

1) Synthesis of Compound Glu-PEG₄-TATE:

9.5 mg of Fmoc-Glu(Boc)-OH (Fmoc and Boc protected glutamic acid), 25 μL of diisopropylethylamine (DIPEA) and 5 μL of diethylphosphonium chloride (DECP) were added to a 20 mL glass vial containing 27.5 mg PEG₄-TATE (a commercially available tumor targeting peptide dissolved in 2.6 mL dimethylformamide (DMF)), after mixed and dissolved, the mixed solution was stirred at room temperature for 2 h, then subjected to LC-MS analysis. Results showed that the Fmoc and Boc protected amino condensation polypeptide product was obtained, recorded as intermediate compound I; then 0.6 mL piperidine was added to the intermediate compound I solution and stirred at room temperature for 1 h, to remove the Fmoc protecting group of intermediate compound I and give the product Glu-PEG₄-TATE. After purification by HPLC and lyophilization, about 17.5 mg of pure compound was obtained, with a yield of 68%.

2) Synthesis of Compound NOTA-2PEG₄-TATE:

17.5 mg of the compound Glu-PEG4-TATE obtained in step 1) dissolved in 2 mL of dimethyl sulfoxide (DMSO), 20 μL DIPEA and 24.0 mg NOTA-PEG4-NHS (1,4,7-triazacyclononane-N,N′,N″-triacetic acid activated ester, 2 equivalents) were mixed and dissolved. The mixture was stirred at room temperature for 20 min and the reaction process was monitored by HPLC. After the compound Glu-PEG₄-TATE in the above step was consumed, the generation of Boc-protected NOTA-PEG₄-TATE was detected by LC-MS. Then 0.1 mL of trifluoroacetic acid (TFA) was added to remove the protecting group Boc to obtain a target compound formed by linking glutamic acid to the PEG4 segment respectively, simply represented as NOTA-2PEG₄-TATE. The mixture obtained by lyophilization and removal of the solvent DMSO was separated and purified by HPLC, and after lyophilization, about 5.5 mg of target product was obtained, with a yield of 25.6%.

3) Synthesis of Compound NOTA-3PEG4-TATE-RGD:

In a 20 mL glass reaction vial containing 6.5 mg of NOTA-2PEG₄-TATE (dissolved in 1 mL DMSO) obtained in the step 2), 6.0 mg PEG₄-RGD and 10 μL DIPEA were added to mix and dissolve. The mixture was stirred at room temperature for 1 h and purified by HPLC. Purification conditions: diluted with 4 mL of water and preparative HPLC (with C18 column) was applied to inject samples in two portions, and purified at a flow rate of 12 mL/min according to the following gradient. The eluent ingredients: Buffer A: 0.1% TFA in H₂O; Buffer B 0.1% TFA in CH₃CN; gradient elution: 0-20 min: 20-50% Buffer B. After lyophilization, about 1.5 mg of product was obtained, with a yield of 13.5% and a purity of more than 97%. The product was identified by LC-MS: [(MHH)/2]⁺⁺=2930.14, and the calculated (m/z) was 2930.92 (C₁₃₁H₁₉₃N₂₇O₄₃S₃). The target product NOTA-Bn-p-SCN-PEG₄-Glu{PEG₄-Cyclo[Arg-Gly-Asp-(D-Tyr)-Lys]}-PEG₄-(D-Phe)-Cys-Tyr-(D-Trp)-Lys-Thr-Cys-Thr was determined.

Embodiment 2

A TATE-RGD dual-target radioactive molecular probe comprising a TATE polypeptide, an RGD polypeptide and a radionuclide ⁶⁸Ga, wherein the TATE and RGD polypeptides are linked by a PEG₄ molecule to form a TATE-3PEG₄-RGD polypeptide and ⁶⁸Ga is linked by a NOTA. The TATE-RGD dual-target radioactive molecular probe is ⁶⁸Ga-NOTA-3PEG₄-TATE-RGD.

The method of preparing ⁶⁸Ga-NOTA-3PEG₄-TATE-RGD dual-target radioactive molecular probe, comprising the following steps:

(1) ⁶⁸Ga Rinsing

5 mL of 0.1 mol/L HCl was drawn by a 5 mL syringe to elute the germanium-gallium generator slowly, at the same time, the eluent was collected to a 1.5 mL EP tube, 1 mL per tube, a total of 5 tubes. Radioactivity determination was performed for each EP tube, and the most deactivated tube was used for labeling;

(2) Labeling of ⁶⁸Ga-NOTA-3PEG4-TATE-RGD

1.25 mol/L sodium acetate solution was added to a 1 mL⁶⁸Ga eluent, the pH of solution was adjusted to 4.0-4.5, then mixed well, and 20 μg of NOTA-3PEG₄-TATE-RGD prepared in the Embodiment 1 was added to mix well, heated to 100° C. and kept for 10 min; after the reaction was completed, the reaction solution was cooled to room temperature, and 4 mL of sterile water for injection was added, and then filtered to a sterile product bottle through a sterile filter membrane (0.22 pm, 13 mm);

(3) Quality Control

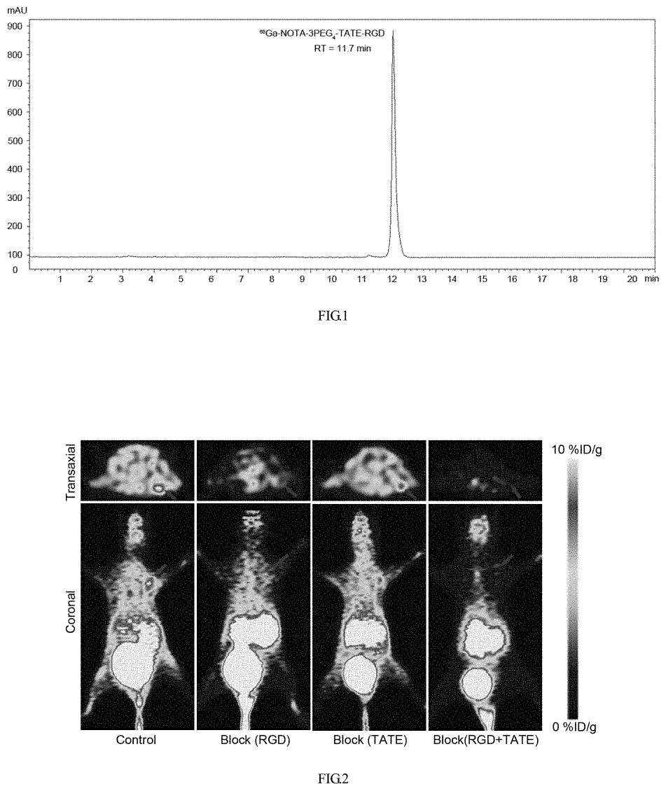

HPLC conditions: C18 column (4.6 mm×250 mm), mobile phase A: E 0.1% trifluoroacetic acid, mobile phase B: water (0.1% trifluoroacetic acid), and flow rate of 1 mL/min: 0˜5 min, mobile phase A 5%;10 min, mobile phase A: 80%; 15 min, mobile phase A: 100%; 18 min, mobile phase A: 100%; 20 min, mobile phase A 5%. The UV detection wavelength was 210 nm and the column temperature was 20° C. Radioactive detection was performed by a dedicated radioactive detector for HPLC. HPLC analysis showed that the retention time of ⁶⁸Ga-NOTA-3PEG₄-TATE-RGD was 11.6 min (FIG. 1), and the radiochemical purity was >99%, without requiring further purification.

(4) Determination of In Vitro Stability

After ⁶⁸Ga-NOTA-3PEG₄-TATE-RGD labeling, fetal bovine serum was added and the radiochemical purity was determined by HPLC at 60 and 120 min, respectively. After determination, the radiochemical purity was 98% and 97%, respectively.

The above TATE-RGD dual-target probe ⁶⁸Ga-NOTA-3PEG₄-TATE-RGD was further prepared into a colorless transparent injection for imaging test or imaging diagnosis. The specific experiments and effects were as follows:

\hat{1} MicroPET Imaging of Small Cell Lung Cancer H69 Tumor-Bearing Mice

The tumor-bearing mice inoculated with H69 tumor subcutaneously in the right forelimb were injected with ⁶⁸Ga-NOTA-3PEG₄-TATE-RGD injection in Embodiment 2 by tail veins at 100-200 μCi for 60 min, and then 10 min static image was collected by a Siemens Inveon micro PET (FIG. 2, wherein the arrow indicated the tumor site). The results showed that, when the probe of Embodiment 2 was injected alone, there was significant radioactivity uptake at the tumor site, the tumor was clearly visible, and the tumor uptake value was 9.78±2.77; while the co-injection of the unlabeled precursor RGD, TATE and RGD+TATE was effective in reducing tumor uptake to 8.23±1.08, 1.41±0.73, and 1.05±0.13, respectively; when concurrently injected with unlabeled precursors RGD and TATE, the radioactivity uptake of tumor was nearly reduced to the background. Small cell H69 tumors were predominantly expressed by SSTR, and the concentration of the dual-target molecular probe of the present invention in tumors was mainly inhibited by unlabeled TATE.

\hat{2} MicroPET Imaging of Non-Small Cell Lung Cancer A549 Tumor-Bearing Mice

The tumor-bearing mice inoculated with A549 tumors in the right forelimb were injected with ⁶⁸Ga-NOTA-3PEG₄-TATE-RGD of injection of embodiment 2 by tail veins at 100-200 μCi for 60 min, and then 10 min static image was collected by a Siemens Inveon micro PET (FIG. 3, wherein the arrow indicated the tumor site). The results showed that, when the probe of Embodiment 2 was injected alone, there was significant radioactivity uptake at the tumor site, the tumor was clearly visible, and the tumor uptake value was 6.46±0.59; while the co-injection of the unlabeled precursor RGD, TATE and RGD+TATE was effective in reducing tumor uptake to 1.75±0.53, 3.80±0.48 and 1.35±0.26, respectively; when concurrently injected with unlabeled precursors RGD and TATE, the radioactivity uptake of tumor was nearly reduced to the background. Non-small cell A549 tumors were predominantly expressed by integrin receptor, and the concentration of the dual-target molecular probe of the present invention in tumors was mainly inhibited by unlabeled RGD.

\hat{4} Biodistribution of ⁶⁸Ga-NOTA-3PEG₄-TATE-RGD in Healthy Mice

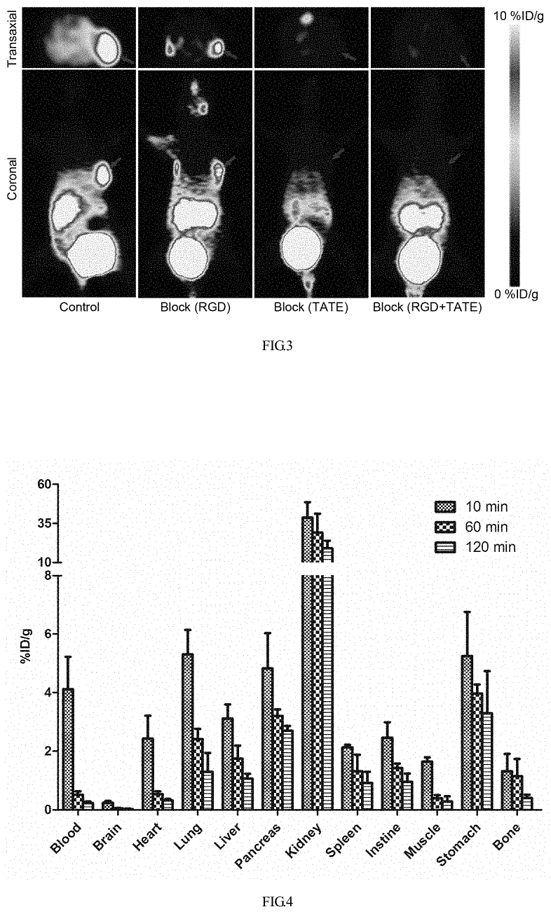

The distribution of healthy Balb/c was shown in FIG. 4, and ⁶⁸Ga-NOTA-3PEG4-TATE-RGD of Embodiment 2 was cleared from the blood, heart and liver faster; the kidney was more radioactive, indicating that the molecular probe was mainly excreted through the kidneys. There was small amount of uptake in the heart, liver and lungs, which decreased quickly over time. The imaging agent had a small amount of distribution in the stomach, intestines, spleen, and pancreas, and little distribution in the brain tissues, indicating that it could not pass through the blood-brain barrier.

\hat{5} Imaging of ⁶⁸Ga-NOTA-3PEG₄-TATE-RGD in Patients with Small Cell Lung Cancer

The ⁶⁸Ga-NOTA-3PEG₄-TATE-RGD PET/CT images of clinically diagnosed small cell lung cancer patients were shown in FIG. 5. ⁶⁸Ga-NOTA-3PEG4-TATE-RGD 3.5 mCi was injected intravenously, 30 min later, the images of truck were collected by Siemens Biograph64 PET/CT, 3 min/bed, and a total of 5 beds were collected. The lesions were clearly displayed and the highest standard uptake value of tumor (SUVmax) was 18.2 (FIG. 5 left). However, the existing single-target imaging agent only showed an increase in the ⁶⁸Ga-NOTA-RGD uptake of the tumor region, and the SUVmax value was 4.7 (FIG. 5 right).

\hat{6} Imaging of ⁶⁸Ga-NOTA-3PEG₄-TATE-RGD in Patients with Non-Small Cell Lung Cancer

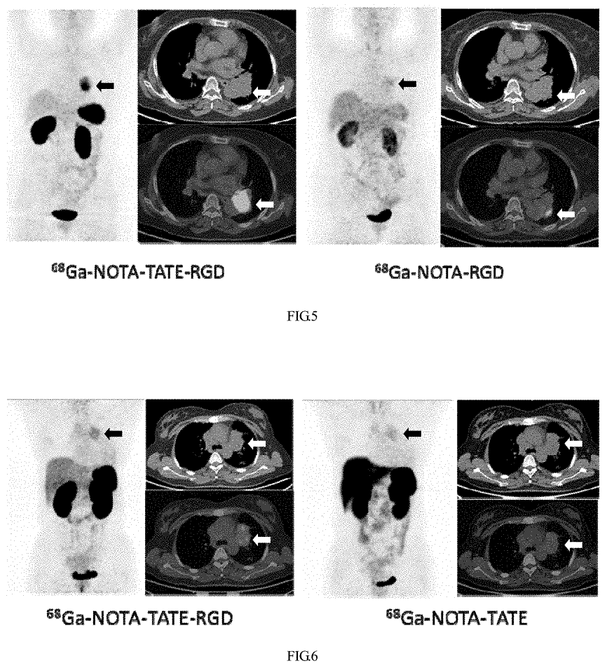

The images of clinically diagnosed non-small cell lung cancer patients were shown in FIG. 6. ⁶⁸Ga-NOTA-3PEG₄-TATE-RGD 3.0 mCi was injected intravenously, 30 min later, the images were collected by Siemens Biograph64 PET/CT, 3 min/bed, and a total of 5 beds were collected. The lesions were clearly displayed and the highest standard uptake value of tumor (SUVmax) was 3.4 (FIG. 6 left). However, the existing single-target imaging agent showed low tumor uptake of ⁶⁸Ga-NOTA-RGD, and the SUVmax value was 2.8 (FIG. 6 right).

1. A targeting polypeptide compound having dual targets, comprising a TATE cyclic peptide structure, an RGD cyclic peptide structure and a NOTA chelating group, wherein the TATE cyclic peptide structure, the RGD cyclic peptide structure and the NOTA chelating group are respectively linked by a PEG segment having a polymerization degree of 1 to 5 or directly linked to a same glutamic acid molecule; the structure of the polypeptide compound can be simplified as NOTA-PEGₙ-Glu{PEGₘ-TATE}-PEG_{P}-RGD, where m, n and p are an integer from 0 to 5 respectively.

2. The polypeptide compound of claim 1, wherein the TATE cyclic peptide structure, the NOTA chelating group and the RGD cyclic peptide structure are linked to a same glutamic acid molecule by a PEG segment having a degree of polymerization of 2 to 5 respectively.

3. The polypeptide compound of claim 2, wherein the TATE cyclic peptide structure, the NOTA chelating group and the RGD cyclic peptide structure are linked to the same glutamic acid molecule by a PEG₄ segment respectively.

4. The polypeptide compound of claim 1, wherein the TATE cyclic peptide structure and the RGD cyclic peptide structure are linked to two carboxyl terminals of the same glutamic acid molecule by a PEG4 molecular segment respectively to form a stable amide bond; the NOTA chelating group is linked to the amino terminal of the same glutamic acid molecule by a PEG₄ segment; the polypeptide compound is designated as NOTA-3PEG₄-TATE-RGD, and the specific structure thereof is as shown in the formula (I) below:

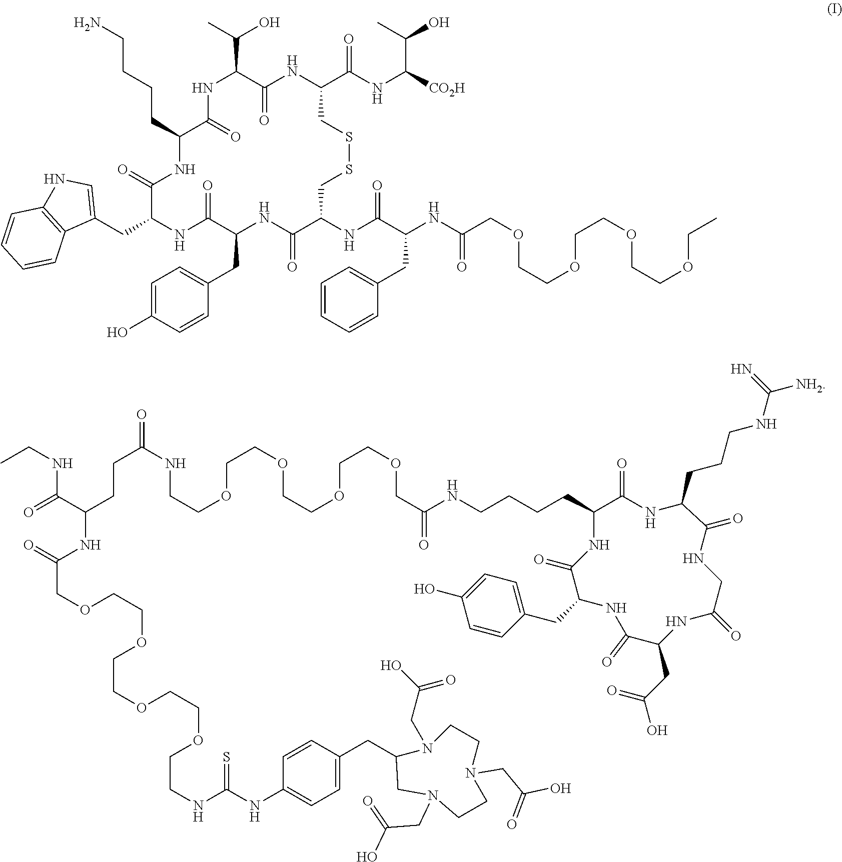

5. A TATE-RGD dual-target radioactive molecular probe, which is a radionuclide-labeled polypeptide complex, wherein the polypeptide complex takes a targeting polypeptide compound having dual targets as a ligand; the targeting polypeptide compound comprises a TATE cyclic peptide structure, an RGD cyclic peptide structure and a NOTA chelating group, wherein the TATE cyclic peptide structure, the RGD cyclic peptide structure and the NOTA chelating group are respectively linked by a PEG segment having a polymerization degree of 1 to 5 or directly linked to a same glutamic acid molecule; the structure of the polypeptide compound can be simplified as NOTA-PEGn-Glu{PEGm-TATE}-PEGP-RGD, where m, n and p are an integer from 0 to 5 respectively.

6. The TATE-RGD dual-target radioactive molecular probe of claim 5, wherein the radionuclide is selected from any one of ⁶⁸Ga, ⁶⁴Cu, ¹⁸F, ⁸⁹Zr or ¹⁷⁷Lu; preferably from any one of 68Ga, 64Cu Or 18F; and most preferably 68Ga.

7. The TATE-RGD dual-target radioactive molecular probe of claim 5, wherein it is a radionuclide ⁶⁸Ga-labeled polypeptide complex, the polypeptide complex takes the targeting polypeptide compound as a ligand, and the dual-target radioactive molecular probe is simply represented as ⁶⁸Ga-NOTA-3PEG₄-TATE-RGD.

8. A method of preparing the targeting polypeptide compound having dual targets of claim 1, comprising the following steps:
a) mixing a protected glutamic acid with polypeptide PEGₙ-TATE in a molar ratio of 1-10:1-10, and carrying out an amino condensation reaction to obtain a first product linked by PEGₙ segment of TATE polypeptide and the protected glutamic acid, where n is an integer from 0 to 5;
b) deprotecting the group Fmoc of the first product obtained in the step a) under the piperidine condition to obtain a second product, simply represented as Glu-PEGₙ-TATE, wherein n is an integer from 0 to 5;
c) reacting the second product obtained in step b) with NOTA-PEGₘ-NHS under DIPEA condition to obtain a third product of Boc-protected glutamic acid that is linked to the NOTA group and the TATE peptide by a PEGₘ segment and a PEGₙ segment respectively, wherein n and m are integers from 0 to 5 respectively;
d) deprotecting the group Fmoc of the third product obtained in the step c) under the TFA conditions to obtain a fourth product of glutamic acid that is linked to the NOTA group and the TATE peptide by a PEG m segment and a PEGₙ segment respectively, simply represented as NOTA-PEGₘ-Glu(PEGₙ-TATE), where n and m are integers from 0 to 5 respectively;
e) reacting the fourth product obtained in step d) with the polypeptide PEGₚ-RGD under the DIPEA condition, where p is an integer of 0 to 5, finally obtaining a tumor targeting polypeptide compound having dual targets NOTA-PEGₙ-Glu{PEGₘ-TATE}-PEG_{P}-RGD.

9. The method of preparing dual- target radioactive molecular probe of claim 7, comprising the following steps:
dissolving the NOTA-3PEG₄-TATE-RGD of claim 4 in deionized water; rinsing germanium-gallium (⁶⁸Ge/⁶⁸Ga) generator into a EP tube with 5 mL of 0.1 mol/L high-purity hydrochloric acid solution, collecting 1 mL of the solution containing the highest content of radioactivity, adding 93 μL of 1.25 mol/L sodium acetate to adjust the pH of the mixture to 4-4.5; adding 20 μg of the precursor to the mixture and mix well, heat to 100 ° C. for 10 min; after completion of the reaction, cooling the reaction solution to room temperature, then adding 4 mL of sterile water for injection, filtering the solution to a sterile product bottle through a sterile filter membrane (0.22 μm, 13 mm).

10. (canceled)
